SEQUENCE LISTING (12 sequences)

SEQ ID NO: 1 - DNA, 20 bp, Artificial sequence
  Synthetic nucleic acid
agaacccggatctgcacaac

SEQ ID NO: 2 - DNA, 22 bp, Artificial sequence
  Synthetic nucleic acid
aggatttgcctagcatcttgct

SEQ ID NO: 3 - DNA, 28 bp, Artificial sequence
  Synthetic nucleic acid
cttcaagctgaactatgaccactttact

SEQ ID NO: 4 - DNA, 24 bp, Artificial sequence
  Synthetic nucleic acid
catggtctggaacttcttctgaga

SEQ ID NO: 5 - DNA, 17 bp, Artificial sequence
  Synthetic nucleic acid
ttccggcaacgctacga

SEQ ID NO: 6 - DNA, 20 bp, Artificial sequence
  Synthetic nucleic acid
tccatccatgaagcctttgg

SEQ ID NO: 7 - DNA, 20 bp, Artificial sequence
  Synthetic nucleic acid
agaacaagctggcccagaaa

SEQ ID NO: 8 - DNA, 21 bp, Artificial sequence
  Synthetic nucleic acid
caccccttcgatccactctct

SEQ ID NO: 9 - DNA, 23 bp, Artificial sequence
  Synthetic nucleic acid
tcctgaccctgaagtatccgata

SEQ ID NO: 10 - DNA, 22 bp, Artificial sequence
  Synthetic nucleic acid
ggtgccagatcttttccatgtc

SEQ ID NO: 11 - DNA, 22 bp, Artificial sequence
  Synthetic nucleic acid
gccgctagaggtgaaattcttg

SEQ ID NO: 12 - DNA, 21 bp, Artificial sequence
  Synthetic nucleic acid
cattcttggcaaatgctttcg

(19) United States
(12) Patent Application Publication
(10) Pub. No.: US 2015/0125428 A1
(43) Pub. Date: May 7, 2015

(54) Multipotent Vascular Stem Cells and Methods of Use Thereof

(71) Applicant: The Regents of the University of California, Oakland, CA (US)

(72) Inventors: Song Li, Orinda, CA (US); Zhenyu Tang, Berkeley, CA (US); Aijun Wang, Berkeley, CA (US)

(73) Assignee: The Regents of the University of California, Oakland, CA (US)

(21) Appl. No.: 14/375,059

(22) PCT Filed: Jan. 29, 2013
(86) PCT No.: PCT/US13/23647

§ 371 Date: Jul. 28, 2014

Related U.S. Application Data
(60) Provisional application No. 61/594,174, filed on Feb. 2, 2012.

(51) Int. Cl. A61K 35/44; C12N 5/071

(52) U.S. Cl. CPC A61K 35/44; C12N 5/0692
USPC 424/93.7; 435/325; 435/383

(57) ABSTRACT

A substantially enriched mammalian multipotent vascular stem cell (MVSC) population is provided, as well as compositions comprising the population. Methods are provided for the isolation, purification, and culture of the MVSCs. The MVSCs are useful in various applications, which are also provided.

### CROSS-REFERENCE

This application claims the benefit of U.S. Provisional Patent Application No. 61/594,175, filed Feb. 2, 2012, which application is incorporated herein by reference in its entirety.

### STATEMENT REGARDING FEDERALLY SPONSORED RESEARCH

This invention was made with government support under Grants No. R01 HL083900 and EB12240 awarded by The National Institutes of Health. The government has certain rights in the invention.

### BACKGROUND

Vascular diseases are a leading cause of death in many countries. The development of vascular diseases, such as intimal hyperplasia and atherosclerosis, involves both vascular and inflammatory cells. Vascular smooth muscle cells (SMCs) are a major cell type in the tunica media of the blood vessel wall, and heterogeneous phenotypes of SMCs have been observed upon vascular injury. A widely accepted dogma is that the de-differentiation of contractile SMCs into proliferative and synthetic SMCs plays an important role in vascular remodeling and disease development, as evidenced by the decreased expression of contractile markers, including smooth muscle myosin heavy chain (SM-MHC) and calponin-1 (CNN1), in proliferative and synthetic SMCs. Further studies suggest that SMCs have diverse embryological SMC origins, such as neural crest and mesoderm, and that only a subpopulation of SMCs may de-differentiate and participate in neointima formation.

### LITERATURE

U.S. Patent Publication No. 2007/0202086

### SUMMARY

A substantially enriched mammalian multipotent vascular stem cell (MVSC) population is provided, as well as compositions comprising the population. Methods are provided for the isolation, purification, and culture of the MVSCs. The MVSCs are useful in various applications, which are also provided.

### Features

The present disclosure provides a multipotent vascular stem cell (MVSC) isolated from a mammalian vascular tissue, wherein the MVSC does not express cell markers of differentiated, mature smooth muscle cells such as smooth muscle myosin heavy chain, and wherein the MVSC can differentiate into ectoderm lineage cells or mesoderm lineage cells. In some embodiments, the mammalian vascular tissue is a blood vessel. For example, the blood vessel can be a carotid artery, a carotid vein, an aorta, an abdominal artery, an inferior vena cava, a femoral artery, a jugular vein, or a femoral vein. In some embodiments, the MVSC exhibits telomerase activity.

In some embodiments, the MVSC can differentiate into Schwann cells and peripheral neurons. In some embodiments, the MVSC can differentiate into osteoblasts, chondrocytes, adipocytes, and smooth muscle cells.

In some embodiments, a subject MVSC is characterized by: a) expression of one or more of Sox10, Sox17, neurofilament medium protein, and S100β; b) substantially no expression of CD146, Sca1, CD31, VE-cadherin, CD34, CD133, C-kit, Flk-1, CNN1, and smooth muscle myosin heavy chain; and c) telomerase activity.

The present disclosure provides an enriched cell population comprising multipotent vascular stem cells (MVSCs), wherein at least 50% of the cells in the cell population are MVSCs characterized by: a) expression of one or more of Sox10, Sox17, NFM, and S100β; b) substantially no expression of CD146, Sca1, CD31, VE-cadherin, CD34, CD133, C-kit, Flk-1, CNN1, and smooth muscle myosin heavy chain; and c) telomerase activity.

The present disclosure provides a composition comprising a subject isolated MVSC in a pharmaceutically acceptable carrier. The present disclosure provides a composition comprising a subject population of MVSC cells in a pharmaceutically acceptable carrier. In some cases, the pharmaceutical carrier comprises one or more of a buffer, a surfactant, an antioxidant, a hydrophilic polymer, a dextrin, a chelating agent, a suspending agent, a solubilizer, a thickening agent, a stabilizer, a bacteriostatic agent, a wetting agent, and a preservative.

The present disclosure provides a cell matrix comprising: a) a subject population of MVSC cells; and b) a biocompatible substrate.

The present disclosure provides a synthetic blood vessel comprising: a) a subject isolated MVSC or a subject enriched MVSC population; and b) a matrix. For example, the matrix can comprise polytetrafluoroethylene (PTFE), extended PTFE, polyurethanes, polyethylene terephthalate (PET), a polyamide, a polyimide, a silicone, fluoroethylypolypropylene (FEP), or a polypropylfluorinated amine (PFA).

The present disclosure provides a method of isolating a population of MVSCs, the method comprising: culturing a sample of mammalian vascular tissue; and isolating cells in the culture that do not express cell markers of differentiated smooth muscle cells. In some cases, the method further comprises selecting for one or more positive MVSC cell markers and/or de-selecting for one or more negative MVSC cell markers. In some cases, the cells are maintained in culture for a period of time prior to said selecting. The period of time can be at least 12 hours.

The present disclosure provides a method of repairing a blood vessel in an individual, the method comprising introducing into said individual an effective number of subject isolated MVSCs.

The present disclosure provides a method of repairing a diseased, injured, or defective blood vessel in an individual, the method comprising replacing the diseased, injured, or defective portion of the blood vessel with a subject synthetic blood vessel. For example, the diseased blood vessel can be an atherosclerotic blood vessel, a partially occluded blood vessel, or a totally occluded blood vessel. In some cases, the injured blood vessel is injured as a result of a surgical treatment or a trauma.

### BRIEF DESCRIPTION OF THE DRAWINGS

FIGS. 1A-1W depict characterization of non-contractile cells isolated from rat carotid arterial tunica media. FIGS. 1A-C show cells that were isolated from arterial tunica media using the enzymatic digestion method. The derived cells were immunostained for smooth muscle α-actin (SMA), SM-MHC, CNN1 and Ki67 after being cultured in DMEM with 10% fetal bovine serum (FBS) for 3 days. The arrow in FIG. 1B indicates a non-contractile cell. The arrows in FIG. 1C indicate proliferating non-contractile cells in culture. The arrowhead in FIG. 1C indicates a non-proliferative mature SMC. FIGS. 1D-1F show cells that were isolated from arterial tunica media using the tissue explant culture method. The derived cells were immunostained for SMA, Ki67, SM-MHC and CNN1 after being cultured in DMEM with 10% FBS for 3 days. FIGS. 1G-1I show F-actin staining with FITC-phalloidin for the isolated non-contractile cells cultured in DMEM with 10% FBS for 5 days, 15 days and 30 days. Nuclei were stained with DAPI. FIGS. 1J-1K show real time quantitative PCR was used to detect the gene expression of SMA and CNN1 of isolated non-contractile cells cultured in DMEM with 10% FBS for 5 days, 15 days and 30 days. 18S was used to normalize the relative expression levels. Data are shown as average ±standard deviation (n=3). * indicates significant difference between indicated groups (p<0.01). FIGS. 1L-1S show immunostaining of isolated non-contractile cells cultured in DMEM with 10% FBS for 3 days for various markers, including Sox10, Sox17, Sox1, Snail, vimentin, nestin, the β subunit of neural protein S100 (S100β) and neural filament-medium polypeptide (NFM). FIGS. 1T-1W show flow cytometry analysis of non-contractile cells derived from arterial tunica media cultured in DMEM with 10% FBS for 3 days with CD29, CD44, CD146 and Sca-1 antibodies. Filled grey curves represent negative control samples, red curves represent samples stained with antibodies for CD29 (FIG. 1T), CD44 (FIG. 1U), CD146 (FIG. 1V) or Sca-1 (FIG. 1W). Scale bars are 100 μm.

FIGS. 2A-2J depict results for a differentiation assay, single cell cloning and telomerase activity assay of non-contractile cells derived from rat carotid arterial tunica media. FIGS. 2A-F show staining of differentiated cells cultured from non-contractile cells: Schwann cells for glial fibrillary protein (GFAP) (FIG. 2A), neurons for neuron-specific class IIIβ tubulin (TUJ1) (FIG. 2B), SMCs for SM-MHC (FIG. 2C), chondrocytes for alcian blue (FIG. 2D), adipocytes for oil red (FIG. 2E) and osteoblasts for alizarin red (FIG. 2F). Scale bars in FIGS. 2A-2C are 50 μm. Scale bars in FIGS. 2D-2F are 100 μm. FIGS. 2G-2H show immunostaining of cloned MVSCs for Sox10 and Sox17. FIG. 21 shows telomerase activity assay of MVSCs and the tissues from which MVSCs were isolated. The data are shown as average±standard deviation (n=3). * indicates significant difference between MVSCs and the tissue from which the cells were derived (p<0.05). † indicates significant difference between inferior vena cava and other blood vessels (p<0.05). AO: aorta, CA: carotid artery, JV: jugular vein, AA: abdominal artery, IVC: inferior vena cava, FA: femoral artery, FV: femoral vein. FIG. 2J shows DNA microarray analysis of MVSCs derived from rat carotid artery and jugular vein (n=3).

FIGS. 3A-3O depict characterization of MVSCs derived from a mouse blood vessel by using a lineage tracing model with SM-MHC and Wnt1 as markers. FIG. 3A shows immunostaining of cross sections of carotid artery of SM-MHC-Cre/LoxP-EGFP mouse for enhanced green fluorescent protein (EGFP) and SMA. Arrows indicate non-contractile cells inside tunica media. Scale bar is 50 μm. FIGS. 3B-3C show flow cytometry analysis for EGFP of cells derived from carotid artery of SM-MHC-Cre/LoxP-EGFP mice by using enzymatic digestion (FIG. 3B) (n=3) and cells cultured in DMEM with 10% FBS for 10 days after enzymatic digestion (FIG. 3C) (n=3). FIGS. 3D-3E show phase contrast and fluorescent images of a tissue explant culture from carotid artery of SM-MHC-Cre/LoxP-EGFP mice. Scale bars are 100 μm. FIG. 3F shows flow cytometry analysis for EGFP of cells derived from carotid artery of SM-MHC-Cre/LoxP-EGFP mice using tissue explant culture (n=6). FIGS. 3G-3H show immunostaining of the EGFP-cells derived from carotid artery of SM-MHC-Cre/LoxP-EGFP mice for Sox10 (FIG. 3G) and Sox17 (FIG. 3H). Scale bars are 100 μm. FIGS. 3I-3O show staining of differentiated cell cultured from EGFP-cells: Schwann cells for GFAP and S100β (FIG. 3I), neurons for TUJ1 and Peripherin (FIG. 3J), chondrocytes for alcian blue (FIG. 3K), adipocytes for oil red (FIG. 3L) and osteoblasts for alizarin red (FIG. 3M). Scale bars of FIGS. 3I-3J are 50 μm. Scale bars of FIGS. 3K-3M are 100 μm. FIGS. 3N-3O show X-Gal staining of MVSCs derived from carotid artery (FIG. 3N) and jugular vein (FIG. 3O) of Wnt1-Cre/LoxP-lacZ mice. Scale bars are 50 μm.

FIGS. 4A and 4B depict spontaneous differentiation of MVSCs into mesenchymal stem cell (MSC)-like cells and SMCs cultured in DMEM with 10% FBS. FIG. 4A shows cells cultured for 5, 20 and 60 days, and immunostained for Sox17, CNN1, SM-MHC and Sox10. Scale bar is 100 μm. Cell nuclei were stained with DAPI. FIG. 4B shows a schematic illustration of the spontaneous differentiation of MVSCs and the multipotency of MVSCs at different stages.

FIGS. 5A-F depict differential response of MVSCs and MSC-like cells to the treatment of vascular growth factors. The undifferentiated MVSCs (cultured in DMEM with 10% FBS for 5 days) and MSC-like cells (cultured in DMEM with 10% FBS for 20 days) were treated with 10 ng/ml basic fibroblast growth factor (bFGF), 10 ng/ml PDGF-B or 10 g/ml transforming growth factor-β1 (TGF-β1) for 24 hrs. FIG. 5A shows the results of EdU staining used to quantify the proliferating cells. FIGS. 5B-5F show the results of quantitative PCR analysis used to quantify the gene expression of Sox17 (FIG. 5B), Sox10 (FIG. 5C), aggrecan (FIG. 5D), SMA (FIG. 5E) and CNN1 (FIG. 5F). 18S was used to normalize the relative expression levels. Data are shown as average±standard deviation (n=3). * indicates significant difference between growth factors treated groups and untreated MVSCs (p<0.05). † indicates significant difference between growth factor treated and untreated MSC-like cells (p<0.05). ‡ indicates significant difference between MVSCs and MSC-like cells (p<0.05).

FIGS. 6A-6L depict MVSC activation in vitro and in vivo. FIGS. 6A-6C show immunostaining of the isolated MVSCs by using enzymatic digestion cultured in DMEM with 10% FBS for 24 hours (hrs) (FIG. 6A) and 48 hrs (FIGS. 6B-6C) with SMA, Ki67 and Sox10 antibodies. Scale bar is 100 μm. FIGS. 6D-6L show immunostaining of cross-sections from native carotid arteries (FIGS. 6D-6F) and injured carotid arteries (day 5) (FIGS. 6G-6L) with Sox10, SMA and Ki67 antibodies. A: Adventitia, L: Lumen, M: Media. The nuclei were stained with DAPI. Scale bars are 50 μm.

FIGS. 7A-7O show that MVSCs are the major cell type in neointima. FIGS. 7A-7F show immunostaining of cross-sections from carotid arteries at 15 days (FIGS. 7A-7C) and 30 days (FIGS. 7D-7F) for Sox10 and Ki67 after vascular injury. I: intima, L: lumen, M: media. Scale bars are 50 μm. FIG. 7G shows cross sections of carotid arteries at 5 weeks post injury that were subjected to alcian blue staining Scale bar is 50 μm. Square indicates the area where the cells were isolated. FIGS. 7H-7I show cells isolated from neointima that were stained for MVSC markers Sox10 and Sox17. FIGS. 7J-7O show cells isolated from neointima that were subjected to differentiation assays and stained for SMC markers SM-MHC and CNN1 (FIG. 7J), Schwann cell markers GFAP and S100β (FIG. 7K), neuronal marker TUJ1 and Peripherin (FIG. 7L), aggrecan in chondrogenic culture (with alcian blue) (FIG. 7M), oil droplets in adipogenic culture (with oil red) (FIG. 7N), and calcified matrix in osteoblastic culture (with alizarin red) (FIG. 7O). Scale bars of 7J-7L are 50 μm. Scale bars of FIGS. 7M-7O are 100 μm. The nuclei were stained with DAPI.

FIG. 8 is a table showing expression of various cell markers in the subject MVSCs.

FIG. 9 is a table showing expression of various cell markers in the subject MVSCs.

### DEFINITIONS

As used herein the term “isolated,” with reference to a cell, refers to a cell that is in an environment different from that in which the cell naturally occurs, e.g., where the cell naturally occurs in a multicellular organism, and the cell is removed from the multicellular organism, the cell is “isolated.” An isolated cell can be present in a mixed population of cells, where the population can be said to be “enriched” for the isolated cell. For example, an isolated MVSC cell can be present in a mixed population of cells in vitro, where the mixed population comprising MVSCs and cells that are not MVSCs. An “enriched” population of MVSC is a cell population in which at least about 50%, at least about 60%, at least about 70%, at least about 80%, at least about 90%, at least about 95%, at least about 98%, or more than 98%, of the cells in the cell population are MVSCs.

By “cell surface marker” is meant a protein expressed on the surface of a cell, which is detectable, e.g., via specific antibodies. Cell surface markers that are useful in the present disclosure include, but are not limited to, the CD (clusters of differentiation) antigens CD29, CD31, CD34, CD44, CD133, and CD146.

By “intracellular marker” is meant a gene or gene product, such as a transcription factor, that is detectable within a cell. For example, SOX10 is a transcription factor that can be detected, e.g., via flow cytometry by using fluorescent substrates.

By “positive for expression” is meant that a marker of interest, whether intracellular or extracellular, is detectable in or on a cell using any method, including, but not limited to, flow cytometry (e.g., flow cytometry using a detectably labeled antibody specific for the marker). The terms “positive for expression,” “positively expressing,” “positive,” “expressing,” “+,” and “pos” used in superscript are used interchangeably herein.

By “negative for expression” is meant that a marker of interest, whether intracellular or extracellular, is not detectable in or on a cell using any method, including but not limited to flow cytometry (e.g., flow cytometry using a detectably labeled antibody specific for the marker). The terms “negative for expression,” “negative expressing”, “negative,” “not expressing,” “−,” and “neg” used in superscript are used interchangeably herein.

By “substantially free” is meant that less than about 10%, 9%, 8%, 7%, 6%, 5%, 4%, 3%, 2%, 1%, or even 0% of the cells in the population express the markers of interest.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, a human, a non-human primate, a rodent (e.g., a mouse, a rat, etc.), an ungulate, a canine, a lagomorph, a feline, etc. In some embodiments, a subject of interest is a human. In some embodiments, a subject is a non-human animal such as a non-human primate, a rodent (e.g., a mouse or a rat), or a lagomorph.

A “therapeutically effective amount” or “efficacious amount” means a number of cells that, when administered to a subject for treating a disease, is sufficient to effect such treatment for the disease. The “therapeutically effective amount” will vary depending on the cell, the disease and its severity and the age, weight, etc., of the subject to be treated.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a multipotent vascular stem cell” includes a plurality of such cells and reference to “the cell marker” includes reference to one or more cell markers and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

A substantially enriched mammalian multipotent vascular stem cell (MVSC) population is provided, as well as compositions comprising the population. Methods are provided for the isolation, purification, and culture of the MVSCs. The MVSCs are useful in various applications, which are also provided.

### Isolated Multipotent Vascular Stem Cells

The present disclosure generally provides a substantially enriched mammalian multipotent vascular stem cell (MVSC) population. The MVSCs present in the enriched population exhibit expression of cell markers, such as Sox10 and Sox1, and can differentiate into ectoderm and/or mesoderm lineage cells. The present disclosure further provides methods of isolating the MVSCs from vascular tissues, and methods of differentiating the MVSCs into ectoderm and/or mesoderm lineage cells.

The present disclosure provides isolated MVSCs. For example, an in vitro cell population enriched in MVSCs is provided. An “enriched” population of MVSC is a cell population in which at least about 50%, at least about 60%, at least about 70%, at least about 80%, at least about 90%, at least about 95%, at least about 98%, or more than 98%, of the cells in the cell population are MVSCs.

### Tissue Sources and Culture Techniques

MVSCs of the present disclosure may be isolated from any of a variety of mammalian vascular tissues, including but not limited to a carotid artery, a carotid vein, an aorta, an abdominal artery, an inferior vena cava, a jugular vein, a femoral artery, or a femoral vein. The source of the vascular tissue can be any of a variety of mammals, including, e.g., rodents (e.g., rats; mice); non-human primates; humans; etc.

MVSCs may be cultured from vascular tissue by utilizing enzymatic digestion or explant culture methods. In the enzymatic digestion technique, a sample of tissue (e.g., a biopsy) is cut into small pieces and incubated under sterile conditions with an enzyme (e.g., collagenase) that digests extracellular matrix proteins. Incubation with the enzyme frees the cells from the extracellular matrix, and the freed cells can then be isolated and re-suspended in a suitable cell culture medium for further manipulation.

In the tissue explant culture technique, a sample of tissue (e.g., a biopsy) is cut into small pieces and the pieces are placed in a suitable cell culture environment (e.g., a petri dish or tissue culture flask containing a suitable culture medium). Over time, cells migrate out of the tissue sample and grow onto the surface of the tissue culture flask. Once the cells have grown onto the surface of the culture flask, they can be expanded and transferred to fresh culture dishes or flasks for further manipulation.

### Cell Markers

The MVSCs of the present disclosure may be characterized by expression of one or more cell markers, including, e.g., cell surface markers, transcription factors, and cytoskeletal proteins. Cell surface markers include the well-known clusters of differentiation (CD) cell surface markers. Antibodies and reagents that specifically bind to these cell surface markers are known in the art and are readily available to the public. Other exemplary cell surface markers include membrane-bound proteins, extracellular binding ligands, and the like.

In addition to cell surface markers, cell markers may also be present within a cell. Such cell markers include, e.g., transcription factors, cytoskeletal proteins, and the like. Antibodies and other reagents that can enter a cell and specifically bind to such cell markers are known in the art and are readily available to the public.

For example, a subject isolated MVSC can express, e.g., one or more of cell surface markers CD29 and CD44, and/or transcription factors Sox10 and Sox1; and may also express cytoskeletal proteins peripherin and S100β. A subject MVSC may also be characterized by a lack of expression of certain markers (e.g., may be negative for certain markers), such as cell surface markers CD146, CD31/PECAM1, VE-cadherin, CD34, CD133, c-kit, and Flk-1. Exemplary cell markers are described in further detail below.

### Positive Cell Markers:

Isolated MVSCs of the present disclosure are generally characterized by positive expression of a variety of cell markers, including neural crest cell markers, neural cell markers, and endoderm markers. Positive cell markers are summarized in Table 1 below.

TABLE 1
Positive cell markers founds in MVSCs.

| Name | Type |
|---|---|
| Sox10 | Transcription factor |
| Sox1 | Transcription factor |
| Snail | Transcription factor |
| Vimentin | Cytoskeletal protein |
| Nestin | Cytoskeletal protein |
| Neurofilament medium protein | Cytoskeletal protein |
| Peripherin | Cytoskeletal protein |
| BRN3a | Transcription factor |
| Phox2b | Transcription factor |
| S100β | Regulatory protein |
| Sox17 | Transcription factor |
| CD29 | Cell surface protein |
| CD44 | Cell surface protein |
| CD73 | Cell surface protein |
| CD90 | Cell surface protein |

### Negative Cell Markers:

MVSCs of the present disclosure may also be characterized by their lack of expression of certain cell markers, including, e.g., various cell lineage markers and markers of mature vascular cell phenotypes, such as mature smooth muscle cells. Negative cell markers are summarized in Table 2 below.

TABLE 2
Negative cell markers in MVSCs.

| Name | Type |
|---|---|
| CD146 | Cell surface marker |
| CD31/PECAM1 | Cell surface marker |
| VE-CADHERIN | Cell surface marker |
| CD34 | Cell surface marker |
| CD133 | Cell surface marker |
| C-KIT | Cell surface marker |
| Flk-1 | Cell surface marker |
| SCA-1 | Cell surface marker |

### Markers of Differentiation:

MVSCs of the present disclosure may also be characterized by their lack of expression of cell markers indicative of terminally differentiated cell phenotypes, such as those provided below in Table 3. MVSCs may also be characterized by their ability to express these cell markers under desired conditions, e.g., when the MVSCs are cultured in an appropriate induction medium that brings about differentiation of the MVSCs.

TABLE 3
Cell markers of Differentiation

| Name | Type |
|---|---|
| GFAP | Schwann cell differentiation |
| TUJ1 | Peripheral neuron |
| | differentiation |
| Aggrecan, detected with alcian blue | Chondrocyte differentiation |
| Lipids, detected with Oil Red | Adipocyte differentiation |
| Mineralization, detected with alizarian red | Osteoblast differentiation |
| SM-MHC | Smooth muscle differentiation |

### Cell Marker Patterns:

MVSCs of the present disclosure can be identified by their expression pattern of cell markers. For example, MVSCs of the present disclosure can be positive for one or more of the following markers: Sox1, Sox10, Sox17, snail, vimentin, nestin, NFM, peripherin, Brn3a, Phox2b, S100β, CD29, CD44, CD73, and CD90; and may be negative for one or more of the following markers: SM-MHC, CD146, Sca-1, CD31, VE-cadherin, CD34, CD133, c-Kit, and Flk-1.

In some embodiments, the subject MVSCs are cells isolated from vascular tissue cultures that are negative for expression of one or more cell markers of mature smooth muscle cell phenotypes, e.g., SM-MHC. In some embodiments, the subject MVSCs are cells isolated from vascular tissue cultures that exhibit positive expression of at least one, two, three, four, five, six, seven, eight, nine, ten, or eleven of the positive cell markers provided in Table 1. In some embodiments, the subject MVSCs are cells isolated from vascular tissue cultures that are negative for expression of at least one, two, three, four, five, six, seven, or eight of the negative cell markers provided in Table 2.

MVSCs of the present disclosure generally display uniform expression of the above-listed positive and negative cell marker patterns. In some embodiments, at least 10%, 20%, 30%, 40%, 50%, 60%, 70%, 80%, 90%, 95%, 96%, 97%, 98%, 99%, up to about 100% of the MVSCs within an isolated population express one or more positive cell markers of interest, while at the same time being substantially free of one or more negative cell markers.

### Cellular Isolation Procedures:

Selective methods known in the art and further described herein may be used to isolate and characterize the subject MVSCs. Typically, a population of cells obtained from vascular tissue is reacted with, e.g., monoclonal antibodies, and enriched populations of cells expressing target cell markers are either positively or negatively selected with, e.g., immunomagnetic beads by complement mediated lysis, agglutination methods, or fluorescence activated cell sorting (FACS). The functional attributes of the resulting subpopulation having a defined cell marker profile may then be determined, e.g., using additional assay techniques.

If desired, unwanted cells may be removed using a negative selection separation step. For example, magnetic bead separations may be used initially to remove large numbers of unwanted cells, e.g., cells that express cell markers that are inconsistent with the desired phenotype of the MVSCs of the present disclosure.

Procedures for cell separation may include, but are not limited to, positive or negative selection by means of magnetic separation using, e.g., antibody-coated magnetic beads, affinity chromatography, cytotoxic agents joined to a monoclonal antibody or used in conjunction with a monoclonal antibody, including, but not limited to, complement and cytotoxins, and “panning” with antibodies attached to a solid matrix, e.g., plate elutriation, or any other convenient technique.

Techniques providing accurate cell separation include, but are not limited to, flow cytometry, which can have varying degrees of sophistication, e.g., a plurality of color channels, low angle and obtuse light scattering detecting channels, impedance channels, and the like. The antibodies for the various cell markers may be illuminated by different fluorochromes. Fluorochromes that may find use in a multicolor analysis include phycobiliproteins, e.g., phycoerythrin and allophycocyanins; fluorescein; and Texas red. The cells may also be selected against dead cells, by employing dyes that selectively accumulate in dead cells (e.g., propidium iodide and 7-aminoactinomycin D (7-AAD)). For example, the cells can be collected in a medium comprising about 2% fetal calf serum (FCS) or 0.2% bovine serum albumin (BSA).

Other techniques for positive selection may be employed, which permit accurate separation, such as affinity columns, and the like. The method of choice should permit the removal of unwanted cells to a residual amount of less than about 50%, less than about 40%, less than about 30%, less than about 20%, less than about 10%, less than about 5%, or less than about 1% of the desired population of MVSCs.

In some embodiments, a multi-step isolation procedure can be used to generate a population of MVSCs. For example, in a first step, cells are positively selected by first sorting for cellular expression of desired cell markers, for example CD29 and/or CD44 by using FACS. In a second step, cells are either positively selected for expression of cell markers such as Sox10, or negatively selected for lack of expression of cell markers, such as CD34, CD133, and CD146. Such multi-step isolation procedures may generally employ a sufficient number of steps to isolate a population of MVSCs exhibiting a desired phenotype, e.g., positive expression of desired cell markers as well as negative expression of unwanted cell markers.

In some embodiments, an isolation procedure may involve the use of a single negative depletion step. In accordance with such a procedure, cells expressing unwanted cell markers are eliminated in a single step employing appropriate techniques, e.g., FACS employing antibodies directed to the unwanted cell markers.

In some embodiments, cells are first isolated from vascular tissues and then subsequently cultured for a period of time prior to isolation of target cells using appropriate reagents. For example, in some embodiments, cells isolated from vascular tissues do not begin to express certain positive cell markers for 24 hours. In such embodiments, cells may be isolated form vascular tissues and cultured for up to about 5, up to about 10, up to about 15, up to about 20, up to about 25, up to about 30, up to about 35, up to about 40, or up to about 45 hours before positive and/or negative selection of cell markers is carried out to isolate the desired population of MVSCs.

In some embodiments, cells are cultured in a well-defined medium (e.g., a chemically-defined medium), and MVSC expansion results in MVSCs becoming a dominant population in the culture.

In some embodiments, cells are purified using a cloning technique, e.g., culturing one cell/well in multiple well plates and expanding the cell population derived from a single cell.

### Additional Characteristics of MVSCs:

In addition to the positive and negative cell marker expression patterns described above, the MVSCs of the present disclosure may be further characterized by their activity in various assays, which are described below.

### Telomerase Activity:

The MVSCs of the present disclosure may generally be characterized by telomerase activity. Telomerase activity is an indicator of a cell's ability to escape senescence, and is a general identifier of stem cell status. MVSCs of the present disclosure are characterized by high levels of telomerase activity compared to control cells. Telomerase activity may be detected, e.g., by using a telomeric repeat amplification protocol (TRAP) combined with real-time detection of amplification products using a Quantitative Telomerase Detection kit (US Biomax Inc., see Materials and Methods section).

### Differentiation Potential:

MVSCs of the present disclosure may be generally characterized by their ability to differentiate into ectoderm or mesoderm cell lineages using, e.g., directed differentiation assays. In order to carry out a directed differentiation assay, MVSCs are cultured for 1-4 weeks in an induction cell culture medium that is specifically designed to induce differentiation into a target cell phenotype. The subject MVSCs may be differentiated into, e.g., Schwann cells, peripheral neurons, smooth muscle cells, chondrocytes, adipocytes, or osteoblasts using the specific induction media described below.

Detailed information relating to directed differentiation assays can be found, e.g., in Wang et al., Derivation of smooth muscle cells with neural crest origin from human induced pluripotent stem cells, Cells Tissues Organs, 2012;195(1-2):5-14, Epub 2011 Oct. 14, which is hereby incorporated by reference in its entirety, and in Wang et al., Induced pluripotent stem cells for neural tissue engineering, Biomaterials 2011 August; 32(22):5023-32, Epub 2011 Apr. 22, which is hereby incorporated by reference in its entirety.

For MVSC differentiation into SMCs, MVSCs on collagen-IV-coated culture dishes are cultured in DMEM with 10% FBS with or without TGF-β1 (10 ng/ml) for 4 weeks. The expression of SMC markers SMA (early stage marker), CNN1 (intermediate stage marker) and SM-MHC (mature SMC marker) are then examined by qPCR, immunoblotting and immunostaining

For chondrogenic differentiation, MVSC pellets are cultured in suspension for 4 weeks in the presence of TGF-β3, dexamethasone and ascorbic acid. The cell pellets are cryo-sectioned and immunostained for collagen-II, or stained for glycosaminoglycans using Alcian blue.

For osteogenic differentiation, MVSCs are seeded at a low density (10³ cells/cm²) and grown for 4 weeks in the presence of β-glycerol phosphate, dexamethasone and ascorbic acid. Then cells are fixed in 4% paraformaldehyde and stained with Alizarin Red to detect calcified matrix.

For adipogenic differentiation, confluent MVSCs are treated with insulin, dexamethasone and isobutylxanthine for 3 weeks, and cells are stained with oil red for lipid and fat deposited by the cells.

For MVSC differentiation into Schwann cells, MVSCs are cultured for 2 weeks in N2 medium supplemented with CNTF, bFGF, dibutyryl-cAMP and neuregulin, and the cells are double-stained for Schwann cell markers S 100β and Schwann-2E. The secretion of myelin basic protein is examined using an ELISA kit as a functional assay of Schwann cells.

For MVSC differentiation into peripheral neurons, MVSCs are cultured for 2 weeks in the presence of BDNF, NGF, GDNF and dibutyryl-cAMP, and the cells are immunostained for peripheral neuron markers peripherin and TUJ1.

Following culture in the appropriate induction medium for an appropriate period of time, the MVSCs can be assessed for the expression of markers of differentiated cell phenotypes. For example, differentiation of MVSCs into Schwann cells can be determined by assaying the cells for expression of GFAP and S100β. Differentiation into peripheral neurons can be determined by assaying the cells for expression of TUJ1 and peripherin. Differentiation into SMCs can be determined by assaying for the expression of SM-MHC. Differentiation into chondrocytes can be determined by staining for glycosaminoglycans, such as aggrecan, using an Alcian blue staining reagent. Differentiation into adipocytes can be determined by staining for lipids using an oil red staining reagent. Differentiation into osteoblasts can be determined by staining for mineralization using an Alzirian red staining reagent.

Induction medium suitable for use with the subject MVSCs may include cell culture medium containing various growth factors, e.g., bFGF and TGF-β1.

### Methods for Inducing MVSC Differentiation:

The subject MVSCs may be differentiated into cells of various lineages by culturing the MVSCs in an appropriate induction medium for a sufficient amount of time to bring about a change in the phenotype of the cells. By “induction medium” in meant any cell growth medium that brings about a change in the phenotype of MVSCs that are cultured in it.

The subject MVSCs are suspended in a desired induction medium and incubated under normal cell culture conditions (e.g., 37° C. in a humidified incubator with an atmosphere of about 5% CO₂) for a designated period of time. The MVSCs may be routinely passaged to maintain the cells in the logarithmic growth phase. Once the desired number of passages has been achieved, or the desired amount of culture time has elapsed, the MVSCs may be assayed for expression of cell markers indicating that the cells have taken on the desired phenotype. Once the target cell phenotype has been achieved, the cells can be used, e.g., in any one of the therapeutic, research, and/or screening methods discussed in more detail herein.

Alternatively, the subject MVSCs may be induced to differentiate into target cell phenotypes based on mechanical stresses in the culture environment, such as, e.g., shear stress and mechanical strain. Alternatively or in addition, the subject MVSCs may be co-cultured with other cells, e.g., feeder cells, which are capable of inducing the MVSCs to differentiate into a target cell phenotype.

### Methods for Maintaining MVSC Multipotency:

The multipotency of the subject MVSCs can be maintained by culturing the cells in a suitable medium that inhibits their differentiation. In some embodiments, the MVSCs are cultured in Dulbecco's modified eagle medium (DMEM) supplemented with 2% chick embryo extract (CEE), 1% fetal bovine serum (FBS), and 20 ng/mL of basic fibroblast growth factor (bFGF) in order to maintain their multipotency. MVSCs can be expanded using this growth medium while maintaining their multipotency.

### Compositions

The present disclosure provides cell compositions comprising a subject isolated MVSC or comprising a subject enriched population of MVSCs.

A subject cell composition comprises a subject isolated MVSC or a subject enriched population of MVSCs; and will in some embodiments comprise one or more further components, which components are selected based in part on the intended use of the MVSCs. Suitable components include, but are not limited to, salts; buffers; stabilizers; protease-inhibiting agents; cell membrane- and/or cell wall-preserving compounds, e.g., glycerol, dimethylsulfoxide, etc.; nutritional media appropriate to the cell; and the like.

The present invention provides compositions, e.g., pharmaceutical compositions, comprising a comprising a subject isolated MVSC or comprising a subject enriched population of MVSCs; and a pharmaceutically acceptable excipient. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

### Cell Matrices

The present disclosure provides a cell matrix comprising a subject isolated MVSC or comprising a subject enriched population of MVSCs.

In some embodiments, a subject composition comprises a subject isolated MVSC, or a subject enriched population of MVSCs, and a matrix (a “subject MVSC/matrix composition”), where a subject MVSC or enriched population of MVSCs is associated with the matrix. The term “matrix” refers to any suitable carrier material to which the MVSC are able to attach or adhere in order to form a cell composite. In some embodiments, the matrix or carrier material is present already in a three-dimensional form desired for later application.

For example, a matrix (also referred to as a “biocompatible substrate”) is a material that is suitable for implantation into a subject. A biocompatible substrate does not cause toxic or injurious effects once implanted in the subject. In one embodiment, the biocompatible substrate is a polymer with a surface that can be shaped into the desired structure that requires repairing or replacing. The polymer can also be shaped into a part of a structure that requires repairing or replacing. The biocompatible substrate can provide the supportive framework that allows cells to attach to it and grow on it.

Suitable matrix components include, e.g., collagen; gelatin; fibronectin; vitronectin; fibrin; fibrinogen; laminin; a glycosaminoglycan; elastin; hyaluronic acid; a proteoglycan; a glycan; poly(lactic acid); poly(vinyl alcohol); poly(vinyl pyrrolidone); poly(ethylene oxide); cellulose; a cellulose derivative; starch; a starch derivative; poly(caprolactone); polyurethane; poly(hydroxy butyric acid); mucin; and the like. In some embodiments, the matrix comprises one or more of collagen, gelatin, fibronectin, vitronectin, fibrin, fibrinogen, laminin, and elastin; and can further comprise a non-proteinaceous polymer, e.g., can further comprise one or more of poly(lactic acid), poly(vinyl alcohol), poly(vinyl pyrrolidone), poly(ethylene oxide), poly(caprolactone), poly(hydroxy butyric acid), cellulose, a cellulose derivative, starch, and a starch derivative. In some embodiments, the matrix comprises one or more of collagen, gelatin, fibronectin, vitronectin, fibrin, fibrinogen, laminin, and elastin; and can further comprise hyaluronic acid, a proteoglycan, a glycosaminoglycan, or a glycan. Where the matrix comprises collagen, the collagen can comprise type I collagen, type II collagen, type III collagen, type V collagen, type XI collagen, and combinations thereof.

The matrix can be a hydrogel. A suitable hydrogel is a polymer of two or more monomers, e.g., a homopolymer or a heteropolymer comprising multiple monomers. Suitable hydrogel monomers include the following: lactic acid, glycolic acid, acrylic acid, 1-hydroxyethyl methacrylate (HEMA), ethyl methacrylate (EMA), propylene glycol methacrylate (PEMA), acrylamide (AAM), N-vinylpyrrolidone, methyl methacrylate (MMA), glycidyl methacrylate (GDMA), glycol methacrylate (GMA), ethylene glycol, fumaric acid, and the like. Common cross linking agents include tetraethylene glycol dimethacrylate (TEGDMA) and N,N′-methylenebisacrylamide. The hydrogel can be homopolymeric, or can comprise co-polymers of two or more of the aforementioned polymers. Exemplary hydrogels include, but are not limited to, a copolymer of poly(ethylene oxide) (PEO) and poly(propylene oxide) (PPO); Pluronic™ F-127 (a difunctional block copolymer of PEO and PPO of the nominal formula EO₁₀₀-PO₆₅-EO₁₀₀, where EO is ethylene oxide and PO is propylene oxide); poloxamer 407 (a tri-block copolymer consisting of a central block of poly(propylene glycol) flanked by two hydrophilic blocks of poly(ethylene glycol)); a poly(ethylene oxide)-poly(propylene oxide)-poly(ethylene oxide) co-polymer with a nominal molecular weight of 12,500 Daltons and a PEO:PPO ratio of 2:1); a poly(N-isopropylacrylamide)-base hydrogel (a PNIPAAm-based hydrogel); a PNIPAAm-acrylic acid co-polymer (PNIPAAm-co-AAc); poly(2-hydroxyethyl methacrylate); poly(vinyl pyrrolidone); and the like.

The cell density in a subject MVSC/matrix composition can range from about 10² cells/mm³ to about 10⁹ cells/mm³, e.g., from about 10² cells/mm³ to about 10⁴ cells/mm³, from about 10⁴ cells/mm³ to about 10⁶ cells/mm³, from about 10⁶ cells/mm³ to about 10⁷ cells/mm³, from about 10⁷ cells/mm³ to about 10⁸ cells/mm³, or from about 10⁸ cells/mm³ to about 10⁹ cells/mm³.

The matrix can take any of a variety of forms, or can be relatively amorphous. For example, the matrix can be in the form of a sheet, a cylinder, a tube, a sphere, etc.

### Synthetic Blood Vessel

The present disclosure further provides a synthetic blood vessel comprising a subject isolated MVSC or comprising a subject enriched population of MVSCs.

The present disclosure provides a prosthetic blood vessel (also referred to as an “artificial blood vessel” or “synthetic blood vessel”), comprising a matrix generally in a tubular form that defines a lumen through which blood can flow, and a subject MVSC embedded within, or arranged on a surface of, the matrix. The prosthetic blood vessel will have first and second ends. In some embodiments, the first and second ends are configured for suturing to a naturally-occurring (endogenous) blood vessel in an individual. Generally, a subject prosthetic blood vessel is longitudinally bendable.

In some embodiments, at least a portion of the prosthetic blood vessel is configured for access by a needle. For example, in some embodiments, a subject prosthetic blood vessel comprises a transcutaneous access port.

A subject prosthetic blood vessel can have a length of from about 0.25 cm to about 10 cm, e.g., from about 0.25 cm to about 0.5 cm, from about 0.5 cm to about 1.0 cm, from about 1.0 cm to about 1.5 cm, from about 1.5 cm to about 2.0 cm, from about 2.0 cm to about 3.0 cm, from about 3.0 cm to about 4.0 cm, from about 4.0 cm to about 5 cm, from about 5 cm to about 7 cm, or from about 7 cm to about 10 cm.

The inner diameter and outer diameter of a subject prosthetic blood vessel are generally compatible with the inner and outer diameters of a natural blood vessel to which the subject prosthetic blood vessel is attached. For example, the inner diameter can range from about 5 mm to about 25 mm, from about 6 mm to about 10 mm, or from about 8 mm to about 20 mm. The outer diameter can range from about 5 mm to about 25 mm, from about 6 mm to about 10 mm, or from about 8 mm to about 20 mm.

A subject isolated MVSC, or a subject enriched population of MVSC, is/are in some embodiments disposed on an inner surface of the tubular matrix of a subject prosthetic blood vessel, e.g., on an inner surface that defines a lumen through which blood flows. A subject isolated MVSC, or a subject enriched population of MVSC, is/are in some embodiments disposed between a first layer and a second layer of the tubular matrix.

In some embodiments, the matrix is a single layer. In other embodiments, the matrix is provided in two or more layers. For example, in some embodiments, an external support layer is included, where the external support layer comprises a knit, tubular mesh capable of expanding radially to accommodate radial expansion within normal compliance range.

A subject prosthetic blood vessel is able to withstand ordinary hemodynamic pressures without leaking or rupturing. For example, a subject prosthetic blood vessel is capable of resilient radial expansion in a manner mimicking the compliance properties of an artery. For example, the compliance of subject prosthetic blood vessel can from 3%/100 mm Hg to 30%/100 mm Hg, where compliance is expressed as percentage change in the internal diameter of a vessel per a 100 mm Hg change in vessel pressure.

The matrix comprises one or more biocompatible materials. Exemplary suitable materials include, e.g., polytetrafluoroethylene (PTFE); extended (or expanded) PTFE; a polymer sold under the trademark GORE-TEX; polyethylene terephthalate (PET); ultra thin wall (UTW) material ranging in thickness from about 0.08 millimeter to about 0.25 millimeter; regular thin wall material (RTW) ranging in thickness from about 0.3 millimeter to about 0.8 millimeter; polyamides; polyimides; silicones; polyurethanes; polyesters; fluoroethylypolypropylenes (FEP); polypropylfluorinated amines (PFA); other fluorinated polymers; and the like.

The matrix can comprise a substance that promotes cell attachment, e.g., fibrin glue, combinations of fibrinogen and thrombin, collagen, basement membrane, alginate, and mixtures of two or more of the foregoing.

A subject prosthetic blood vessel can comprise, in addition to a subject isolated MVSC, or a subject enriched population of MVSC, one or more additional agents. Suitable agents include, e.g., an analgesic, an anesthetic, an antimicrobial compound, an antibody, an anticoagulant, an antifibrinolytic agent, an anti-inflammatory compound, an antiparasitic agent, an antiviral compound, a cytokine, a cytotoxin or cell proliferation inhibiting compound, a chemotherapeutic drug, a growth factor, an osteogenic or cartilage inducing compound, a hormone, an interferon, a lipid, an oligonucleotide, a polysaccharide, a protease inhibitor, a proteoglycan, a polypeptide, a steroid, a vasoconstrictor, a vasodilator, a vitamin, and a mineral.

A subject prosthetic blood vessel can be used as a carotid bypass graft; as an arterio-venous (A-V) shunt; as a coronary artery bypass graft; to replace a portion of a diseased coronary artery; to replace a portion of a diseased peripheral artery or vein; to replace a portion of a defective peripheral artery or vein; to replace a portion of a defective coronary artery; to replace or bypass an atherosclerotic artery; etc. Exemplary uses of a subject synthetic blood vessel include aneurysm repair, trauma repair, cardiovascular disease treatment, and the like.

### Utility

MVSCs of the present disclosure find use in various therapeutic, research, and drug screening applications, which are discussed in detail below.

### Therapeutic Uses

MVSCs of the present disclosure find use in various therapeutic applications. For example, the subject MVSCs may be used to repair, reconstitute, or reconstruct damaged or diseased tissues in a subject. Such damage or disease may be caused by genetic or environmental conditions, e.g., heart disease, etc. Autologous cells or allogeneic cells may be used for such purposes. MVSCs may also be used, e.g., in the construction of vascular grafts and/or other structures useful in replacing or repairing defective, diseased, damaged, or otherwise compromised tissue within a subject.

MVSCs may be administered in any physiologically acceptable medium, e.g., intravascularly, although they may also be introduced into another convenient site, where the cells may find an appropriate attachment site for regeneration and differentiation. The cells may be introduced by injection, catheter, or the like. The cells may be frozen at liquid nitrogen temperatures and stored for long periods of time prior to use, being capable of use upon thawing. If frozen, the cells can be stored in a 10% dimethylsulfoxide (DMSO), 50% fetal calf serum (FCS), 40% RPMI 1640 medium. Once thawed, the cells may be expanded by use of growth factors and/or stromal cells associated with MVSC proliferation and differentiation.

Genes may be introduced into the MVSCs for a variety of purposes, e.g., to prevent vascular disease, to replace genes having a loss of function mutation, to introduce genes having a desired function, to suppress activation of a particular response, etc. Alternatively, vectors may be introduced that express antisense mRNA or ribozymes, thereby blocking expression of an undesired gene. Other methods of gene therapy include the introduction of drug resistance genes to enable MVSCs, and cells derived from them, to have an advantage and be subject to selective pressure, for example the multiple drug resistance gene (MDR), or anti-apoptosis genes, such as bcl-2. Various techniques known in the art may be used to transfect the MVSCs, e.g., electroporation, calcium precipitated DNA, fusion, transfection, lipofection, and the like. The particular manner in which the DNA is introduced is not critical to the practice of the subject methods.

Many vectors useful for transferring exogenous genes into target cells are available. The vectors may be episomal, e.g., plasmids, virus derived vectors such cytomegalovirus, adenovirus, etc., or may be integrated into the target cell genome, through homologous recombination or random integration, e.g., retrovirus-derived vectors such MMLV, HIV-1, ALV, etc.

### Research Applications

The subject MVSCs find use in a variety of research applications. For example, the nutrient medium from a culture of MVSCs, which is a conditioned medium, may be isolated at various stages of culture and the components analyzed. Separation can be achieved, e.g., using HPLC, reversed phase-HPLC, gel electrophoresis, isoelectric focusing, dialysis, or other non-degradative techniques that allow for separation by molecular weight, molecular volume, charge, combinations thereof, or the like. One or more of these techniques may be combined to further enrich for specific fractions and/or components.

The MVSCs may be used in conjunction with a culture system to isolate and evaluate factors or compounds associated with the differentiation and maturation of MVSCs and/or other cell types. Thus, the MVSCs may be used in assays to determine the activity of growth media, such as conditioned media, to evaluate fluids for growth factor activity, involvement with dedication of cell lineages, or the like.

### Drug Screening

The subject MVSCs can be used in drug screening applications, e.g., for identifying candidate agents that modulate the activity of MVSCs, for identifying candidate agents that modulate proliferation of MVSCs, etc. A wide variety of assays may be used for this purpose, including immunoassays for protein binding; determination of cell growth; differentiation and functional activity; and the like.

For example, examination of gene expression in the subject MVSCs based on exposure to candidate agents can be examined. The expressed set of genes may be compared between MVSCs cultured under different conditions, or against other types of cells known in the art. For example, in order to determine the genes that are regulated following exposure of the MVSCs to a candidate agent, one could compare the set of genes expressed in an MVSC culture exposed to a candidate agent to that of an MVSC culture not exposed to the candidate agent.

Any suitable qualitative or quantitative methods known in the art for detecting specific mRNAs can be used. mRNA can be detected by, for example, hybridization to a microarray, in situ hybridization in tissue sections, by reverse transcriptase-PCR, or in Northern blots containing poly A mRNA. One of skill in the art can readily use these methods to determine differences in the size or amount of mRNA transcripts between two samples. For example, the level of particular mRNAs in an induced MVSC culture is compared with the expression of the mRNAs in a reference sample, e.g., a non-induced MVSC culture.

In another screening method, a test sample of MVSCs is assayed for the level of an analyte of interest based on exposure to a candidate agent. Analytes of interest may include, e.g., intracellular proteins, transcription factors, cell surface markers, mRNA molecules, and the like. Analysis can be accomplished using any of a number of methods to determine the target analyte in the test sample. For example, cells can be permeabilized to stain cytoplasmic molecules. In general, antibodies that specifically bind an analyte of interest are added to a sample and incubated for a period of time sufficient to allow binding to the epitope. The antibody can be detectably labeled for direct detection (e.g., using radioisotopes, enzymes, fluorescers, chemiluminescers, and the like), or can be used in conjunction with a second stage antibody or reagent to detect binding (e.g., biotin with horseradish peroxidase-conjugated avidin, a secondary antibody conjugated to a fluorescent compound, e.g. fluorescein, rhodamine, Texas red, etc.) The absence or presence of antibody binding can be determined by various methods, including flow cytometry of dissociated cells, microscopy, radiography, scintillation counting, etc. Any suitable alternative methods of qualitative or quantitative detection of levels or amounts of differentially expressed target analytes can be used, for example ELISA, western blot, immunoprecipitation, radioimmunoassay, Northern blot, Southern blot, in situ hybridization, etc.

In some embodiments, a subject screening assay identifies agents that reduce the proliferation or differentiation of an MVSC. Agents that reduce the proliferation and differentiation of an MVSC by at least about 10%, at least about 15%, at least about 20%, at least about 25%, or more than 25%, are considered candidate agents for reducing neointima formation in a blood vessel.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Materials and Methods:

The following materials and methods were used throughout the examples provided herein.

### Generation of Transgenic Mice and Genotyping:

The smooth muscle myosin heavy chain (SM-MHC)-Cre transgenic mice (B6.Cg-Tg(Myh11-cre,-EGFP)2Mik/J) and loxP-enhanced green fluorescent protein (EGFP) transgenic mice (Gt(ROSA)26Sortm1(rtTA,EGFP)) were obtained from Jackson Labs. The two mouse lines were crossed to generate SM-MHC-Cre/loxP-EGFP bitransgenic mice. Genotyping was performed with polymerase chain reaction (PCR) by using total DNA derived from mice tail tissues as described previously.

### Cell Isolation:

The cell isolation methods were described previously. Briefly, the segments of carotid artery, jugular vein, aorta, abdominal artery, inferior vena cava, femoral artery and femoral vein were collected from Sprague Dawley (SD) rats or mice and washed three times with phosphate buffered saline (PBS) supplemented with 1% penicillin/streptomycin (P/S). The surrounding connective tissues and adventitia were dissected away. Endothelium was removed by scraping off the cell layer on the luminal surface with sterile scalpel blades. For tissue explant culture methods, the tunica media were cut into mm-size and placed onto the surface coated with 1% CellStart (Invitrogen Corp.) in 6-well plates. The cells were cultured in Dulbecco's Modified Eagle Medium (DMEM) (Invitrogen) with 10% fetal bovine serum (FBS) (Thermo Fisher Scientific Inc.), or in DMEM with 2% chick embryo extract (CEE) (MP Biomedical, Inc.), 1% FBS, 1% N2 (Invitrogen Corp.), 2% B27 (Invitrogen Corp.), 100 nM retinoic acid (RA) (Sigma-Aldrich, Inc.), 50 nM 2-mercaptoethanol (2ME) (Sigma-Aldrich, Inc.), 1% Penicillin/Streptomycin and 20 ng/ml basic fibroblast growth factor (bFGF) (R&D Systems, Inc.) (Maintenance medium). Cells started migrating out of the tissue explants within 2 days. For enzymatic digestion methods, tissues were incubated with 3 mg/ml type II collagenase (Sigma-Aldrich Inc.) in DMEM with a 1/5 (w/v) ratio of tissue (g) to enzyme solution (ml). After incubation at 37° C. for 30 min, the same volume of 1 mg/ml elastase (Sigma-Aldrich, Inc.) solution was added to the solution containing the tissue and collagenase. The tissues were incubated for another 1-2 hours until all the tissues were digested. Cells were then seeded onto CellStart-coated dishes and maintained at 37° C. in an incubator with 5% CO₂. The medium was changed every other day.

### Cell Cloning, Sphere Formation and Differentiation Assays:

For the clonal assays, multipotent vascular stem cells (MVSCs) were detached, and the cells were resuspended with maintenance medium and filtered through membranes with 40-μm pore size to obtain single cells. Filtered cells were seeded onto CellStart-coated 96-well plates at the clonal density (1 cell/well) and cultured for 3 weeks at 37° C. in an incubator with 5% CO₂.

For sphere formation assay, MVSCs were dissociated and plated onto a 6-well ultra-low-attachment plates (Corning, Inc.) at the density of 0.5×10⁶ cells/well in the presence of maintenance medium for 1 week. The derived neural sphere-like aggregates were collected and embedded into OCT compound (Tissue Tek Inc.) for cryosectioning and immunostaining.

For the directed differentiation of MVSCs into peripheral neurons, Schwann cells, osteoblasts, adipocytes and chondrocytes, the cells were incubated in specific induction media described previously for 1-3 weeks.

### Telomerase Activity Assay

Telomerase activities of MVSCs and the tissues from which the cells were isolated were detected by using telomeric repeats amplification protocol (TRAP) combined with real-time detection of amplification products using a Quantitative Telomerase Detection kit (US Biomax Inc.) according to the instruction of the manufacturer. Total protein extract (0.5 μg for each sample) was used in each reaction. Real-time polymerase chain reaction (PCR) analysis was performed by using an ABI PRISM 7000 Sequence Detection System. The amount of molecules was quantified by using the standard curve, and normalized with the level of α-actin in each sample measured by an enzyme-linked immunosorbent assay (ELISA) kit (Cell Signaling Technology, Inc.).

### Growth Factor Treatment and Cell Proliferation Assay:

Undifferentiated MVSCs and partially differentiated MVSCs (cultured in DMEM/10% FBS for 15 days) were starved in DMEM with 1% FBS for 24 hr followed by the treatment of 10 ng/ml basic fibroblast growth factors (bFGF) (Peprotech, Inc.), 10 ng/ml platelet derived growth factors-B (PDGF-B) (Peprotech, Inc.) or 10 ng/ml transforming growth factor-β1 (TGF-β1) (Peprotech, Inc.) for another 24 hr. The cell proliferation was quantified by using Click-iT EdU Alexa Fluor 488 HCS Assay kit (Invitogen Corp.) according to the manufacturer's instructions. Briefly, the cells were incubated with 10 μM-ethynyl-2′-deoxyuridine (EdU) for 1 hr. Then cells were washed with PBS and fixed with 4% paraformaldehyde (PFA) followed by incubation with detection solution as well as 4′,6-diamidino-2-phenylindole (DAPI).

RNA Isolation, Oligonucleotide Microarray, and qPCR:

MVSCs derived from carotid arteries and jugular veins were lysed with Trizol reagent (Invitrogen Corp.) and total RNA was extracted by using chloroform and phenol extractions and precipitated by using isopropanol, and the resulting RNA pellet was washed with 75% ethanol. For microarray analysis, the RNA pellet was re-suspended in 12 μl of nuclease-free H₂O and analyzed with a UV spectrophotometer to confirm purity by using the ratio of absorption A 260/A 280(≧1.80). RNA concentration was determined by using the RiboGreen quantification assay (Molecular Probes, Inc.), and the samples subsequently were diluted to a concentration of 0.50 mg/ml. Then, 10 μl of each sample was used for the analysis with an Affymetrix oligonucleotide microarray U133AA of Av2 chip containing 31099 probe sets. Samples were labeled and hybridized according to Affymetrix protocols. Signal intensities were obtained for all probe sets and were organized by using GeneTraffic version 3.2 microarray analysis software (Iobion).

For qPCR, RNA pellets were resuspended in 20 μl of diethyl pyrocarbonate (DEPC)-treated H₂O and were quantified as described above. cDNA was synthesized by using two-step reverse transcription with the ThermoScript RT-PCR system (Invitrogen, Inc), followed by qPCR with SYBR green reagent and the ABI Prism 7000 Sequence Detection System (Applied Biosystems). Primers for the genes of interest were all designed by using the ABI Prism Primer Express software version 2.0 (Applied Biosystems). The sequences of the primers are as follows:

Soxl7-5′:
(SEQ ID NO: 1)
5′-AGAACCCGGATCTGCACAAC-3′
Sox17-3′:
(SEQ ID NO: 2)
5′-AGGATTTGCCTAGCATCTTGCT-3′
Aggrecan-5′:
(SEQ ID NO: 3)
5′-CTTCAAGCTGAACTATGACCACTTTACT-3′
Aggrecan-3′:
(SEQ ID NO: 4)
5′-CATGGTCTGGAACTTCTTCTGAGA-3′
SM-MHC-5′:
(SEQ ID NO: 5)
5′-TTCCGGCAACGCTACGA-3′
SM-MHC-3′:
(SEQ ID NO: 6)
5′-TCCATCCATGAAGCCTTTGG-3′
CNN1-5′:
(SEQ ID NO: 7)
5′-AGAACAAGCTGGCCCAGAAA-3′
CNN1-3′:
(SEQ ID NO: 8)
5′-CACCCCTTCGATCCACTCTCT-3′
SMA-5′:
(SEQ ID NO: 9)
5′-TCCTGACCCTGAAGTATCCGATA-3′
SMA-3′:
(SEQ ID NO: 10)
5′-GGTGCCAGATCTTTTCCATGTC-3′
18S-5′:
(SEQ ID NO: 11)
5′-GCCGCTAGAGGTGAAATTCTTG-3′
18S-3′:
(SEQ ID NO: 12)
5′-CATTCTTGGCAAATGCTTTCG-3′.

RNA from rat MVSCs, rat contractile smooth muscle cells (SMCs), or rat osteosarcoma cells was used to create standard curves for each gene, and the gene expression level of each sample was normalized by the expression level of 18S ribosomal RNA of the respective sample. Data were analyzed by using ABI Prism 7000 SDS software (Applied Biosystems).

### Rat Carotid Artery Endothelial Denudation Model:

All experimental procedures with animals were approved by the ACUC committee at UC Berkeley, and were carried out according to the institutional guidelines. The rat carotid artery injury model was described previously. Briefly, adult SD rats weighing 200 to 300 g were anesthetized with isoflurane and placed in a prone position. Endoluminal injury to the left common carotid artery was produced by a custom-made wire probe that consisted of a 10-cm-long stainless steel wire soldered with a copper-beaded tip (1.0 mm in diameter). Rats were sacrificed at 5, 15, and 30 days after injury, with 6 animals for each time point. The blood vessels samples were rinsed with PBS and embedded in OCT compound for histological analysis.

### Staining and Histological Analysis:

For immunostaining, cell or tissue sections of blood vessels were fixed with 4% PFA, permeabilized with 0.5% Triton-100 (Sigma-Aldrich, Inc.), and blocked with 1% bovine serum albumin (BSA) (Sigma-Aldrich, Inc.). For actin cytoskeleton staining, samples were incubated in fluorescein isothiocyanate (FITC)-conjugated phalloidin (Invitrogen Corp.) for 30 min to stain filamentous actin (F-actin). For staining of other cell markers, samples were incubated with specific primary antibodies (Supplementary Table 1) for 2 hours in room temperature (RT), washed with PBS for 3 times, and incubated with appropriate Alexa 488- and/or Alexa 546-labeled secondary antibodies (Molecular Probes, Inc.). Nuclei were stained with DAPI (Invitrogen Corp.). Fluorescence images were collected by a Zeiss LSM710 confocal microscope.

For organic dye staining, cells or tissue sections were fixed with 4% PFA for 30 min, washed and stained with alizarin red (Sigma-Aldrich, Inc.), alcian blue (Sigma-Aldrich, Inc.), oil red (sigma-Aldrich, Inc.) or Verhoeff's dye (American MasterTech, Inc.) according to the instruction of the manufacturers. Images were collected by a Zeiss Axioskop 2 plus microscope.

### Flow Cytometry Analysis:

For flow cytometry analysis, cells were dissociated after the exposure to 0.2% EDTA for 20 min at room temperature. The cells in suspension were blocked with 1% BSA, incubated with specific primary antibodies, and stained with secondary antibodies. Negative control sample was incubated with a non-specific antibody with the same isotype as the specific primary antibody, and stained with the same secondary antibody. 7-AAD (BD Pharmingen Inc.) was used to exclude dead cells for quantitative analysis. Cells were analyzed by using FACScan flow cytometer (Becton Dickinson Inc.) and FlowJo software (Tree Star, Inc.).

### Statistics:

Data were reported as means±standard deviation, unless otherwise indicated. All experiments were repeated at least three times. Comparisons among values for all groups were performed by one-way analysis of variance (ANOVA). Holm's t test was used for the analysis of differences between different groups. Significance level was set as p<0.05.

### Example 1

### Identification and Characterization of MVSCs in Rat Arterial Tunica Media

SMCs are the major cell type in tunica media. First, the existence of non-contractile cells in the tunica media layer was verified by immunostaining the cross-sections of carotid arteries from Sprague Dawley (SD) rats for smooth muscle α-actin (SMA) and smooth muscle myosin heavy chain (SM-MHC). The majority of the cells inside the elastic lamina layers were mature and contractile SMCs (SMA+/SM-MHC+), while a small population (less than 5%) were non-contractile cells (SM-MHC−).

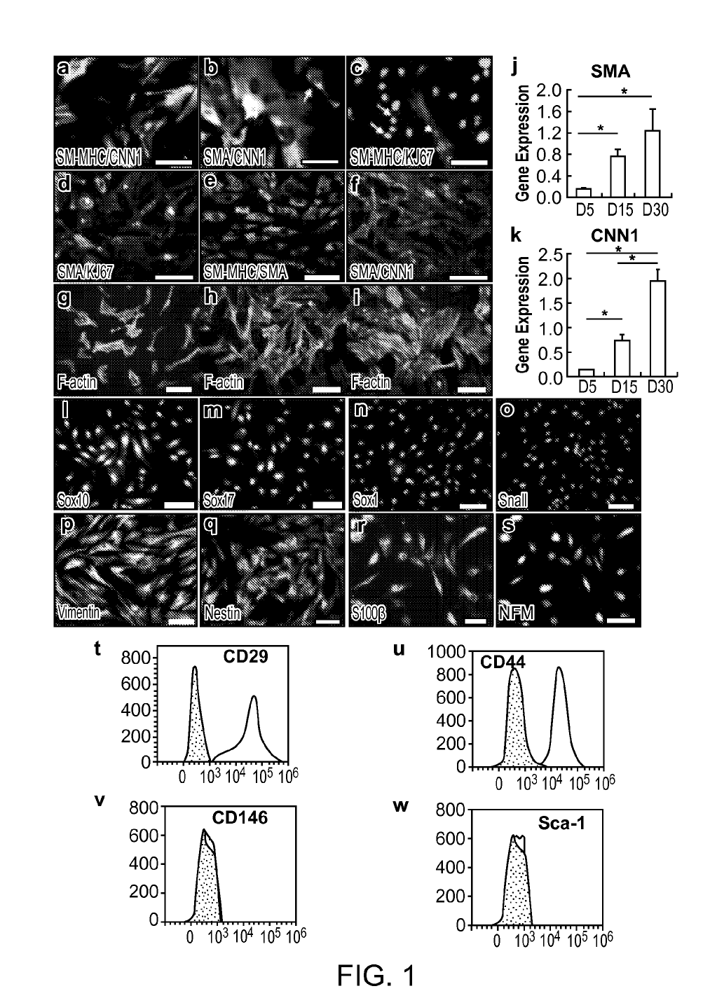

Cells from the tunica media of carotid arteries of SD rats were then isolated and characterized. By using the enzymatic digestion method, a mixed cell population was obtained, including both contractile SMCs and non-contractile cells. The contractile SMCs had SM-MHC and CNN1 assembled into stress fibers and were non-proliferative (negative for Ki67) (FIG. 1, panels a-c). In contrast, the non-contractile cells expressed low level of SMA and were highly proliferative as evidenced by Ki67 staining (FIG. 1, panels b-c). After being cultured in Dulbecco's Modified Eagle's Medium (DMEM) with 10% fetal bovine serum (FBS) for 3 days, the non-contractile cells started to multiply, and eventually dominated the culture.

With the tissue explant culture method, which relies on cell migration, only migratory and proliferative cells can be isolated. The cells migrating out of the tunica media expressed Ki67 and low levels of SMA (FIG. 1, panels d-f). These cells had the same characteristics as the non-contractile cells isolated by using the enzymatic digestion method. In the previous literature, these non-contractile cells were treated as synthetic and/or proliferative SMCs. However, a detailed characterization showed that the cells exhibited significant changes in morphology during the culture for an extended period. The size of cell nucleus and the spreading area of the cells increased significantly (>3 fold) within a 30-day time period, accompanied by the increase of stress fibers (FIG. 1, panels g-i) and the increased expression of SMA and CNN1 (FIG. 1, panels j-k). These results suggest that the synthetic/proliferative SMCs might be derived from the spontaneous differentiation of the non-contractile cell population in the medium with DMEM and 10% FBS.

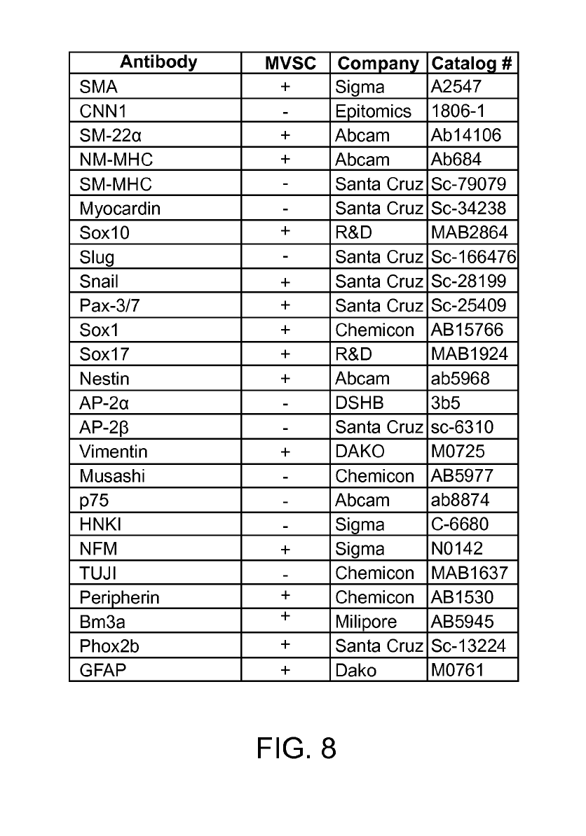

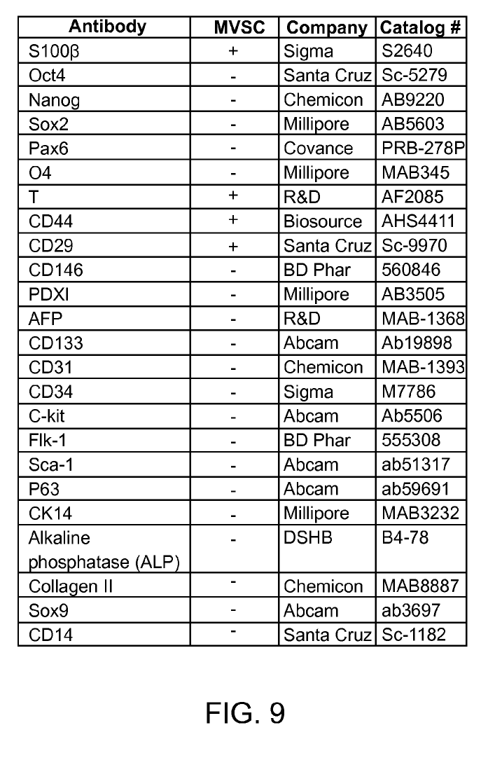

To thoroughly characterize these non-contractile cells at the early stage (day 3) of primary tissue explant culture, protein-marker expression was screened with over 50 antibodies, and the results indicated that isolated non-contractile cells uniformly expressed various markers, including neural crest cell markers Sox10, Sox1, Snail, vimentin and nestin, neural cell makers NFM, peripherin, Brn3a, Phox2b and S100β, endoderm marker Sox17 (FIG. 1, panels 1-s; FIG. 8; FIG. 9), and general MSC markers including CD29 and CD44 (FIG. 1, panels t, u). In addition, these non-contractile cells were negative for CD146 and Sca-1 (FIG. 1, panels v, w), suggesting that these non-contractile cells were distinct from previously identified perivascular MSC-like cells19 and Sca-1+ progenitors. These cells were also negative for markers for EC and EC conversion-derived MSC-like cells, e.g., CD31/PECAM1 and VE-cadherin 23, as well as the endothelial progenitor cell (EPC) and hematopoietic stem cell (HSC) markers CD34, CD133, c-Kit and Flk-1.

To maintain the phenotype of non-contractile cells, a series of screenings for maintenance medium was performed, resulting in the identification of a modified medium for neural crest stem cell (NCSC) culture that could maintain the cell morphology and the expression of aforementioned markers in the non-contractile cells. The maintenance medium contained DMEM with 2% chick embryo extract (CEE), 1% FBS, and 20 ng/ml of basic fibroblast growth factor (bFGF), and was used to expand the cells for further analysis.

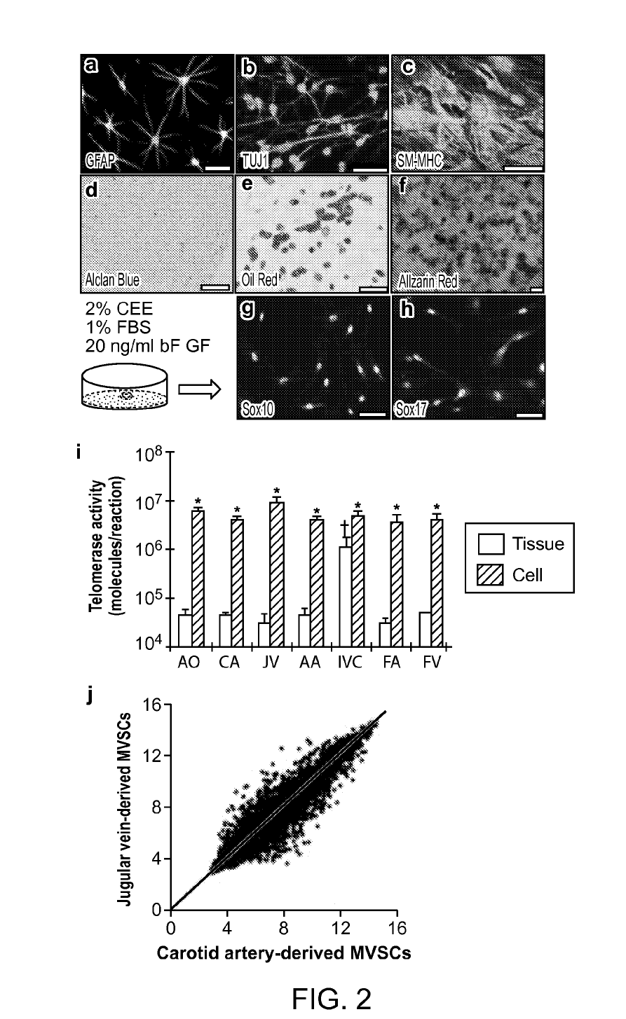

Since these non-contractile cells expressed many markers of NCSCs, the ability to differentiate into ectodermal and mesodermal lineages was determined by treating cells with specific differentiation induction media. Indeed, these non-contractile cells were capable of differentiating into Schwann cells (positive for glial fibrillary acidic protein, GFAP+), peripheral neurons (positive for neuron-specific class III β-tubulin, i.e., TUJ1+), SMCs (SM-MHC+), chondrocytes (alcian blue staining), adipocytes (oil red staining) and osteoblasts (alizarin red staining) (FIG. 2, panels a-f). These results indicated that these non-contractile cells were multipotent, similar to NCSCs. Since these cells also have telomerase activity and can be cloned, they were named MVSCs. MVSCs could be isolated from the tunica media of different blood vessels including jugular vein, aorta, abdominal artery, inferior vena cava, femoral artery and femoral vein beside carotid artery, but not from adventitia layer, endothelium, or fat tissue around blood vessels by using tissue explant culture method.

### Example 2

### Single Cell Cloning and Telomerase Activity Assay

To confirm the stemness of MVSCs, a cloning assay was performed. Dilutional cell cloning assay showed that the average plating efficiency of MVSCs was about 13%, and the cloned MVSCs retained the marker expression including Sox10 and Sox17 (FIG. 2, panels g-h), and could self-renew and maintain multipotency. To determine whether MVSCs had the capability to form neural spheres as neural crest cells, rat MVSCs derived from cloning assay were cultured on ultra-low-attachment plates. Indeed, MVSCs formed neural sphere-like aggregates, and the cells within the spheres retained the expression of the MVSC markers such as Sox17, vimentin, SMA, nestin and S100β.

It was then determined whether blood vessels and isolated MVSCs possess telomerase activity. All blood vessels examined had telomerase activity, suggesting the presence of stem cells in the vascular wall (FIG. 2, panel i). The isolated MVSCs had a marked increase (>100 fold) of telomerase activity compared to the respective vascular tissues from which the cells were isolated, suggesting the enrichment and purification of stem cells from vascular tissues during the cell isolation process. Furthermore, no significant difference was found in telomerase activity in MVSCs isolated from different blood vessels. Interestingly, the telomerase activity of inferior vena cava tissue was significantly higher than that of other blood vessels.

To further compare the MVSCs from different blood vessels, carotid artery and jugular vein were used as representatives. DNA microarray analysis showed that MVSCs from carotid arteries and jugular veins were predominantly identical (FIG. 2, panel j), while only 3.6% of genes in expression showed significant difference of more than 2-fold, suggesting that MVSCs derived from arteries and veins were similar. DNA microarray also showed that MVSCs expressed MSC markers such as CD29, CD44, CD73 and CD90.

### Example 3

### MVSCs were Not Derived from the De-Differentiation of Contractile SMCs

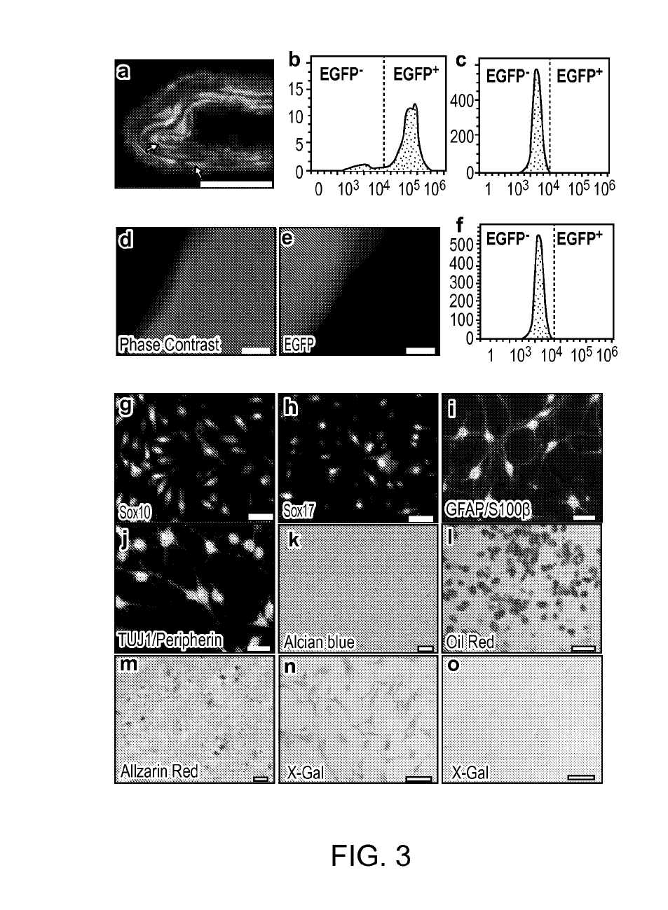

The de-differentiation of SMCs is deemed responsible for the presence of synthetic/proliferative cells. To directly determine whether MVSCs were derived from the de-differentiation of contractile SMCs, lineage tracing was performed by using SM-MHC as a marker. Immunostaining of SMA showed that less than 5% of the cells in the carotid arterial tunica media of the SM-MHC-Cre/loxP-enhanced green fluorescence protein (EGFP) bi-transgenic mice were not labeled with EGFP, indicating the existence of a small population of non-contractile cells that were not derived from SMCs in the tunica media (FIG. 3, panel a), consistent with the observation in the rat model.

With enzymatic digestion culture, the majority (more than 92%) of the cells isolated from the carotid arteries of SM-MHC-Cre/LoxP-EGFP mice were contractile SMCs or contractile SMCs derived cells (EGFP+) (FIG. 3, panel b). However, after being cultured and passaged in DMEM with 10% FBS for 10 days, all the cells in culture were EGFP− (FIG. 3, panel c), indicating that these cells were not derived from contractile SMCs, which was consistent with the previous observation that contractile SMCs were not able to de-differentiate to entry cell cycle in rat model (FIG. 1, panel c).

In addition, by using the tissue explant culture method, cell isolation from the carotid arteries of SM-MHC-Cre/LoxP-EGFP mice showed that the migratory and proliferative cells were negative for EGFP in contrast to strong EGFP fluorescence in the arterial tunica media (FIG. 3, panels d, e). Flow cytometry also showed that none of the derived vascular cells expressed EGFP, which indicated that proliferative and migratory cells isolated from tunica media of blood vessels were not derived from the de-differentiation of contractile SMCs (FIG. 3, panel f). These results provide direct evidence for the first time that contractile SMCs cannot de-differentiate into non-contractile cells and that the synthetic/proliferative cells isolated from blood vessels were not derived from contractile SMCs.

To determine whether these non-contractile cells (EGFP-) were similar to MVSCs identified from rat model, immunostaining and differentiation assays were performed. Immunostaining showed that these non-contractile cells isolated from the carotid artery of SM-MHC-Cre/LoxP-EGFP mice by using tissue explant culture method uniformly expressed MVSC markers including Sox10 and Sox17 (FIG. 3, panels g, h). In addition, these cells could be induced to differentiate into Schwann cells (GFAP+/S100β+), peripheral neurons (Tuj1+/Peripherin+), chondrocytes (alcian blue+), adipocytes (oil red+) and osteoblasts (alizarin red+) by the treatment of specific induction media (FIG. 3, panels i-m), which further confirmed that non-contractile cells in tunica media are MVSCs.

Previous lineage tracing experiments showed that neural crest cells only contributed to the development of carotid artery, aortic arch and large arteries close to the heart. To determine whether MVSCs from different vascular beds were derived from neural crest, MVSCs from different blood vessels were isolated in Wnt1-Cre/LoxP-LacZ mice. MVSCs derived from carotid artery showed positive X-Gal staining while cells derived from jugular vein were negative (FIG. 3, panels n-o), indicating that MVSCs derived from different vascular beds might have different developmental origins.

### Example 4

### MVSCs Spontaneously Differentiated into MSC-Like Cells and SMCs

As shown in FIG. 1, panels g-k and FIG. 2, panel c, MVSCs could differentiate into SMCs, and the cells at various stages of differentiation could contribute to the heterogeneity of SMCs reported previously. It was also possible that previously identified MSC-like cells could be derived from MVSCs. Therefore, spontaneous differentiation of MVSCs was examined by culturing the freshly isolated MVSCs for 8 weeks in DMEM with 10% FBS.

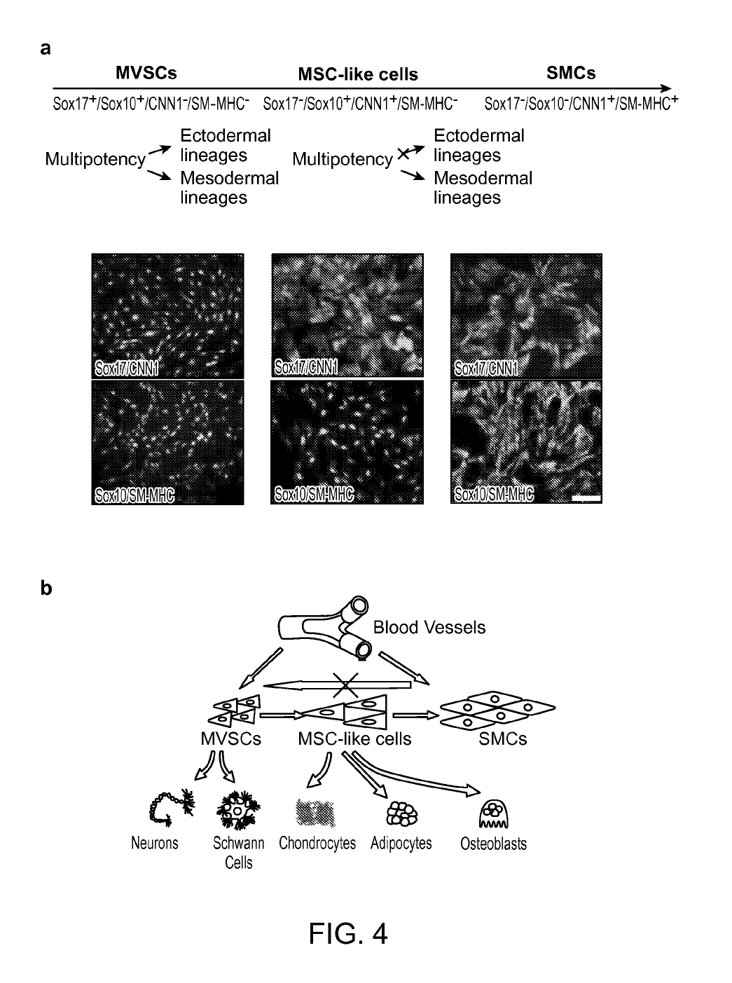

As shown in FIG. 4, panel a, MVSCs were negative for CNN1 and SM-MHC. After 3 weeks, MVSCs expressed CNN1 but lost the nuclear expression of Sox17 (Sox17−/Sox10+/CNN1+/SM-MHC−). At this stage, the cells lost the capability of differentiating into peripheral neurons and Schwann cells, but retained the potential for SMC, osteogenic, chondrogenic and adipogenic differentiation (data not shown; summarized in FIG. 4, panel b), similar to previously reported MSC-like cells. These results suggested that MVSCs could be the precursor of MSC-like cells identified in blood vessel by previous studies.

In addition, after being cultured in DMEM with 10% FBS for 8 weeks, the cells lost the expression of Sox17 and Sox10 and spontaneously differentiated into contractile SMCs (CNN1+/SM-MHC+) (FIG. 4, panel a). These cells also lost the differentiation potential into neural and mesenchymal lineages (summarized in FIG. 4, panel b). These results suggested that long-term culture in the medium containing DMEM with 10% FBS resulted in the spontaneous differentiation of MVSCs into MSC-like cells and subsequently SMCs and that the cells at the various stages of differentiation might explain the heterogeneity of SMCs.

### Example 5

### Differential Response of MVSCs and MSC-Like Cells to Vascular Growth Factors

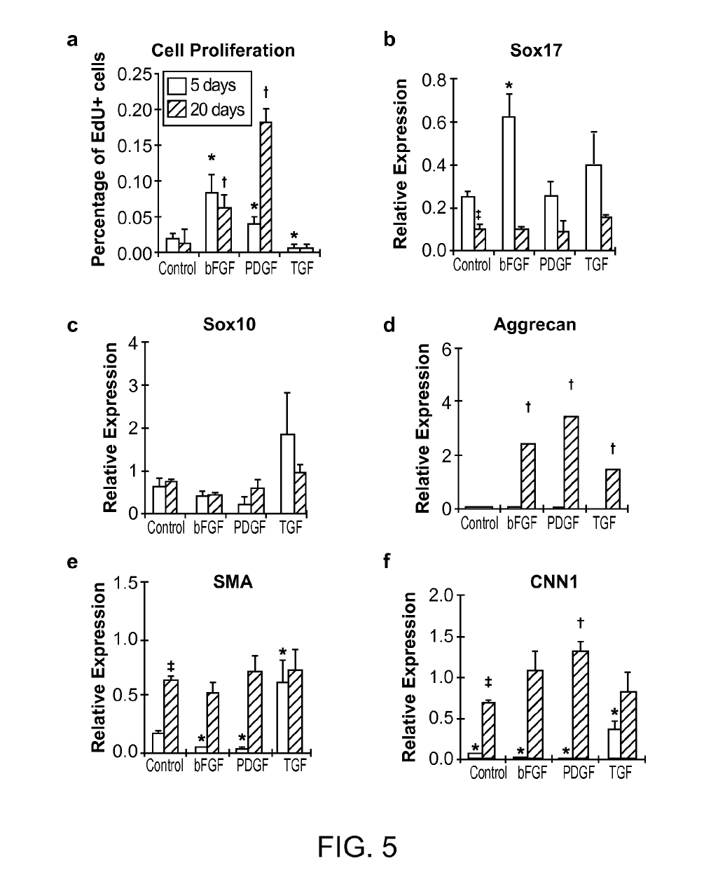

An important question is how MVSCs at different stages of differentiation respond to the same stimulation presented by the vascular microenvironment. To address this issue, the effects of three vascular growth factors on undifferentiated MVSCs (Sox10+/Sox17+) and MVSC-derived MSC-like cells (Sox10+/Sox17−) were compared as an example. The vascular growth factors used in this study included bFGF, platelet derived growth factors-B (PDGF-B) and transforming growth factor-β1 (TGF-β1). PDGF-B and bFGF increased the proliferation of both MVSCs and MSC-like cells, while TGF-β1 suppressed the proliferation of MVSCs (FIG. 5, panel a). It was also noted that PDGF had more dramatic effect on the proliferation of MSC-like cells compared with MVSCs.

Quantitative polymerase chain reaction (qPCR) showed that MSC-like cells had much lower expression of MVSC marker such as Sox17 and higher expression of early SMC markers such as SMA and CNN1 (FIG. 5, panels b, e, f). bFGF significantly increased Sox17 expression while suppressed the expression of SMC markers including SMA and CNN1 of MVSCs (FIG. 5, panels b, e, f), suggesting that bFGF helped maintain MVSCs at undifferentiated state. However, bFGF had no significant effect on the expression of Sox17, SMA and CNN1 but increased the synthesis of aggrecan (FIG. 5, panel d) in MSC-like cells, suggesting that bFGF might promote a synthetic SMC phenotype in MSC-like cells.

PDGF-B also suppressed the expression of SMC markers of MVSCs (FIG. 5, panels e, f), but did not show any effect on Sox17 expression (FIG. 5, panel b). In MSC-like cells, PDGF increased aggrecan synthesis and CNN1 expression, but had no effect on the expression of SMA (FIG. 5, panels d-f). TGF-β1 significantly increased the expression of early SMC markers including SMA and CNN1 of MVSCs (FIG. 5, panels e, f), suggesting that TGF-β1 promoted MVSC differentiation into SMC lineage. In MSC-like cells, TGF-β1 promoted synthetic SMC phenotype by increasing the synthesis of aggrecan (FIG. 5, panel d), similar to bFGF and PDGF. These results demonstrated the MVSCs and MSC-like cells responded differently to vascular growth factors.

### Example 6

### MVSCs can be Activated In Vitro and In Vivo

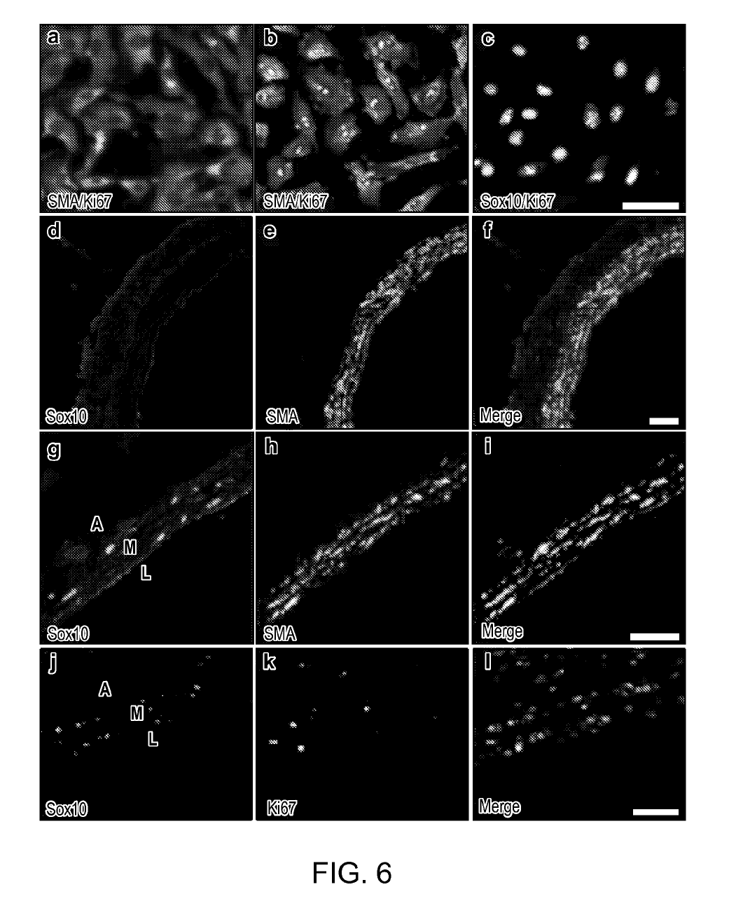

Cell proliferation and expansion are important in the development of vascular diseases. Therefore, in vitro and in vivo studies were performed to determine the transition of MVSCs from quiescent state to proliferative state. MVSCs were first isolated using an enzymatic digestion method. Within 24 hr after cell isolation, immunostaining showed that MVSCs only expressed low levels of SMA, but not Sox10 or Ki67 (FIG. 6, panel a), which indicated that MVSCs were quiescent in the blood vessels. However, after being cultured for another 24 hr, MVSCs started expressing Sox10, as well as Ki67 (FIG. 6, panels b, c), suggesting that MVSCs were activated and became proliferative.

Immunostaining also confirmed that no cells in the normal blood vessel wall expressed MVSC markers (e.g., Sox10, NFM or S100β) or proliferation marker Ki67 (FIG. 6, panels d-f), suggesting that MVSCs were quiescent under normal physiological conditions. Upon vascular denudation injury, less than 5% of cells inside the elastic lamina layers of arterial media were found to express MVSCs markers Sox10, NFM and S100β at day 5 after injury (FIG. 6, panels g-i), and about 40% of Sox10+ cells were positive for proliferative marker Ki67 (FIG. 6, panels j-l). The Ki67+ cells were found only in Sox10+ MVSC population in tunica media, further confirming that MVSCs instead of SMCs could be activated to enter cell cycle upon vascular injury. Of note, Ki67-expressing cells were found in the endothelial layer and adventitia layer after injury, indicating that ECs and adventitial cells were also activated to enter cell cycle for vascular remodeling.

### Example 7

### MVSCs and/or MVSC-Derived Cells are the Major Cell Type in Neointima

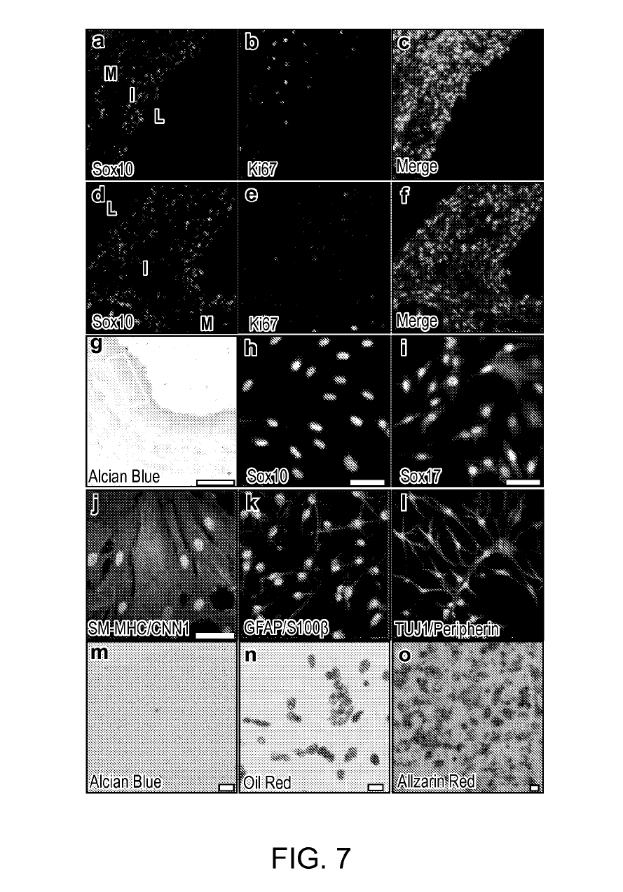

The neointima formation was observed between day 15 and 30 after denudation injury. The majority of the cells in the neointima expressed MVSC makers including Sox10, NFM and S100β (FIG. 7, panels a-f) at day 15 and 30, indicating that MVSCs are the major cell type in neointima. In addition, the percentage of Ki67+ MVSCs inside the neointima was 32% at day 15, and decreased to less than 1% at day 30, suggesting a transient expansion of MVSCs in the vascular wall.

Alcian blue staining and Verhoeff's staining showed significant deposition of collagen I and proteoglycan respectively in neointima populated by MVSCs after 1 month (FIG. 7, panel g), suggesting that MVSCs-derived cells showed the synthetic phenotype and contributed to the matrix synthesis during neointima formation. To determine whether there were still undifferentiated MVSCs in the neointima, cells were isolated from neointima tissue at 1 month after injury by using the explant culture method. Indeed, cells with same marker expression and differentiation potential as MVSCs could be derived from neointima tissues (FIG. 7, panels h-o), suggesting that MVSCs were capable of self-renewal in vivo during vascular remodeling.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A multipotent vascular stem cell (MVSC) isolated from a mammalian vascular tissue, wherein the MVSC does not express cell markers of differentiated, mature smooth muscle cells such as smooth muscle myosin heavy chain, and wherein the MVSC can differentiate into ectoderm lineage cells or mesoderm lineage cells.

2. The MVSC of claim 1, wherein the mammalian vascular tissue is a blood vessel, including but not limited to: a carotid artery, a carotid vein, an aorta, an abdominal artery, an inferior vena cava, a femoral artery, a jugular vein, or a femoral vein.

3. The MVSC of claim 1, wherein the MVSC exhibits telomerase activity.

4. The MVSC of claim 1, wherein the MVSC can differentiate into Schwann cells and peripheral neurons.

5. The MVSC of claim 1, wherein the MVSC can differentiate into osteoblasts, chondrocytes, adipocytes, and smooth muscle cells.

6. The MVSC of claim 1, wherein the MVSC is characterized by:
a) expression of one or more of Sox10, Sox17, neurofilament medium protein, and S100β;
b) substantially no expression of CD146, Sca1, CD31, VE-cadherin, CD34, CD133, C-kit, Flk-1, CNN1, and smooth muscle myosin heavy chain; and
c) telomerase activity.

7. An enriched cell population comprising multipotent vascular stem cells (MVSCs), wherein at least 50% of the cells in the cell population are MVSCs characterized by:
a) expression of one or more of Sox10, Sox17, NFM, and S100β;
b) substantially no expression of CD146, Sca1, CD31, VE-cadherin, CD34, CD133, C-kit, Flk-1, CNN1, and smooth muscle myosin heavy chain; and
c) telomerase activity.

8. A composition comprising the isolated MVSC of claim 1 in a pharmaceutically acceptable carrier.

9. A composition comprising the population of MVSC cells of claim 7 in a pharmaceutically acceptable carrier.

10. The composition of claim 9, wherein the pharmaceutical carrier comprises one or more of a buffer, a surfactant, an antioxidant, a hydrophilic polymer, a dextrin, a chelating agent, a suspending agent, a solubilizer, a thickening agent, a stabilizer, a bacteriostatic agent, a wetting agent, and a preservative.

11. A cell matrix comprising:
a) the population of MVSC cells of claim 7; and
b) a biocompatible substrate.

12. A synthetic blood vessel comprising:
a) the MVSC of claim 1 or the enriched MVSC population of claim 7; and
b) a matrix.

13. The synthetic blood vessel of claim 12, wherein the matrix comprises polytetrafluoroethylene (PTFE), extended PTFE, polyurethanes, polyethylene terephthalate (PET), a polyamide, a polyimide, a silicone, fluoroethylypolypropylene (FEP), or a polypropylfluorinated amine (PFA).

14. A method of isolating a population of MVSCs of claim 1, comprising:
culturing a sample of mammalian vascular tissue; and
isolating cells in the culture that do not express cell markers of differentiated smooth muscle cells.

15. The method of claim 14, further comprising:
selecting for one or more positive MVSC cell markers and/or de-selecting for one or more negative MVSC cell markers.

16. The method of claim 15, wherein the cells are maintained in culture for a period of time prior to said selecting.

17. The method of claim 16, wherein the period of time is at least 12 hours.

18. A method of repairing a blood vessel in an individual, the method comprising introducing into said individual an effective number of MVSCs of claim 1.

19. A method of repairing a diseased, injured, or defective blood vessel in an individual, the method comprising replacing the diseased, injured, or defective portion of the blood vessel with the synthetic blood vessel of claim 12.

20. The method of claim 19, wherein the diseased blood vessel is an atherosclerotic blood vessel, a partially occluded blood vessel, or a totally occluded blood vessel.

21. The method of claim 19, wherein the injured blood vessel is injured as a result of a surgical treatment or a trauma.
